(19) United States
(12) Patent Application Publication
(10) Pub. No.: US 2010/0082110 A1
(43) Pub. Date: Apr. 1, 2010

(54) INTERVERTEBRAL DISC PROSTHESIS

(76) Inventor: Karl P. Belliard, Du Plessis Mace (FR)

Correspondence Address: SPRINKLE IP LAW GROUP, 1301 W. 25TH STREET, SUITE 408, AUSTIN, TX, 78705, US

(21) Appl. No.: 11/993,364

(22) PCT Filed: Jun. 23, 2006
(86) PCT No.: PCT/FR2006/050624

§ 371 Date: Dec. 20, 2007

(30) Foreign Application Priority Data
Jun. 24, 2005 (FR) 05 06443

(51) Int. Cl. A61F 2/44

(52) U.S. Cl. 623/17.16

(57) ABSTRACT

The invention relates to an intervertebral disk prosthesis including a lower element consisting of a lower prosthetic plate and an insert defining a convex spherical cap-shaped surface and adapted to be fitted in the prosthetic plate. The prosthetic plate includes a housing having a bottom and a peripheral rim interrupted by an opening for inserting the insert, the insert having a base adapted to be placed in the housing. The insert and the prosthetic plate include a first fastening means for fastening together in a direction OZ perpendicular to the bottom of the housing the plate and the insertion end of the insert, and a second fastening means including a non-immobile fastening member disposed in the insertion opening and adapted to cooperate with a complementary member of the insert to fasten together in the direction OZ the second end of the insert and the plate.

The present invention relates to an intervertebral disk prosthesis, and to be more precise, to a prosthesis intended to be implanted between two lumbar vertebrae.

In the event of severe degeneration of the intervertebral disk, it is necessary to proceed with its ablation and to substitute an implant or prosthesis for it. To enable this prosthesis to allow some mobility of the two vertebrae concerned, it consists of two functional elements each having one face anchored to a vertebral plate and another face that constitutes one member of a ball-joint element. Thus movements close to natural movements are made possible by the presence of the ball-joint system.

To enable the prosthesis to be implanted, it is necessary to separate the two vertebrae between which the prosthesis is to be disposed. This separation causes certain traumas, in particular of the ligaments that connect the vertebrae together. It is therefore desirable to have intervertebral disk prostheses available that require as small a separation of the vertebrae as possible.

To this end, there are known in the art intervertebral disk prostheses in three or four separate parts. There is usually a lower prosthetic plate, an upper prosthetic plate defining a concave spherical cap, and an insert that the surgeon places in the lower plate, this insert defining the convex spherical cap-shaped surface defining the ball-joint system.

Because the insert can be fitted after the initial implantation of the upper and lower prosthetic plates, the distance by which the vertebrae are separated can be reduced.

However, the fact that the insert is a part that is mechanically separate from the lower plate requires the insert to be mechanically locked to the lower plate in the three main orthogonal directions. It is, however, necessary for this locking system to be easy for the surgeon to use and for it not to lead to any increase in the overall thickness of the intervertebral disk prosthesis, in turn leading to an increase in the separation of the vertebrae.

The object of the present invention is to provide an intervertebral disk prosthesis comprising an upper plate, a lower plate, and an insert mounted in the lower plate that satisfies the conditions set out above.

To achieve the above object, the intervertebral disk prosthesis of the invention comprises

a lower element consisting of a lower prosthetic plate and an insert defining a convex spherical cap-shaped surface and adapted to be fitted in said prosthetic plate and an upper element defining a concave spherical cap-shaped surface, said prosthesis being characterized in that:
said prosthetic plate includes a housing having a bottom and a peripheral rim interrupted by an insertion opening for inserting said insert;
said insert has a base adapted to be placed in said housing, said base having a lower face for cooperating with the bottom of said housing and an upper face on which said convex spherical cap is formed; and
said insert and said prosthetic plate comprising:
   first fastening means for fastening together in a direction OZ perpendicular to the bottom of said housing said plate and the insertion end of the insert when the insert cooperates with the plate; and
   second fastening means comprising a non-immobile fastening member disposed in said insertion opening and adapted to cooperate with a complementary member of the insert to fasten together in said direction OZ the second end of said insert and said plate.

It is clear that because the insert is fastened when it is placed in the lower prosthetic plate and locked in the direction OZ at its insertion end and at its opposite end, effective fastening of the insert relative to the lower prosthetic plate in the direction OZ is achieved.

It should be made clear that in the present text the expression non-immobile member refers either to a deformable member such as a clipping system operative by elastic deformation, for example, or a removable member that is fitted to lock the second end of the insert to the lower prosthetic plate.

Furthermore, it is clear that it is possible for this non-immobile member to have a shape such that it does not lead to any significant increase in the distance that must be provided between the lower prosthetic plate and the upper prosthetic plate to enable the surgeon to fit the insert.

Said first fastening means preferably comprise:
a projection extending from the insertion end of the base of said insert; and
an opening in the end of the rim of the prosthetic plate opposite the insertion opening that opens into said housing and is adapted to cooperate with said projection when the insert is fitted in said housing of said plate.

In a first embodiment said second fastening means comprise an elastically deformable member projecting into the insertion opening of said plate and fastened thereto and a rigid member projecting from the second end of the base of the insert for clipping the insert by cooperation with said elastically deformable member.

It is clear that the locking of the second end of the insert to the lower prosthetic plate is then effected a by clipping action. It is also clear that it is possible for the clipping element fastened to the prosthetic plate to have a sufficiently low height not to increase significantly the distance that must be provided between the two vertebrae so that the surgeon can fit the insert.

In a second embodiment, said second fastening means consist of a removable and elastically deformable member having a median part cooperating with the second end of the base of the insert and two ends adapted to penetrate into openings in the peripheral rim of said prosthetic plate by virtue of elastic deformation.

It is clear that the locking of the insert to the prosthetic plate in the direction OZ is then effected by the surgeon with the aid of an additional and elastically-deformable part that cooperates both with the insert and with the lower prosthetic plate. This removable element then does not lead to any increased thickness liable to increase the distance that must be provided between the two vertebrae to fit the insert.

Other features and advantages of the invention become more apparent on reading the following description of embodiments of the invention given by way of non-limiting example. The description refers to the appended drawings, in which:

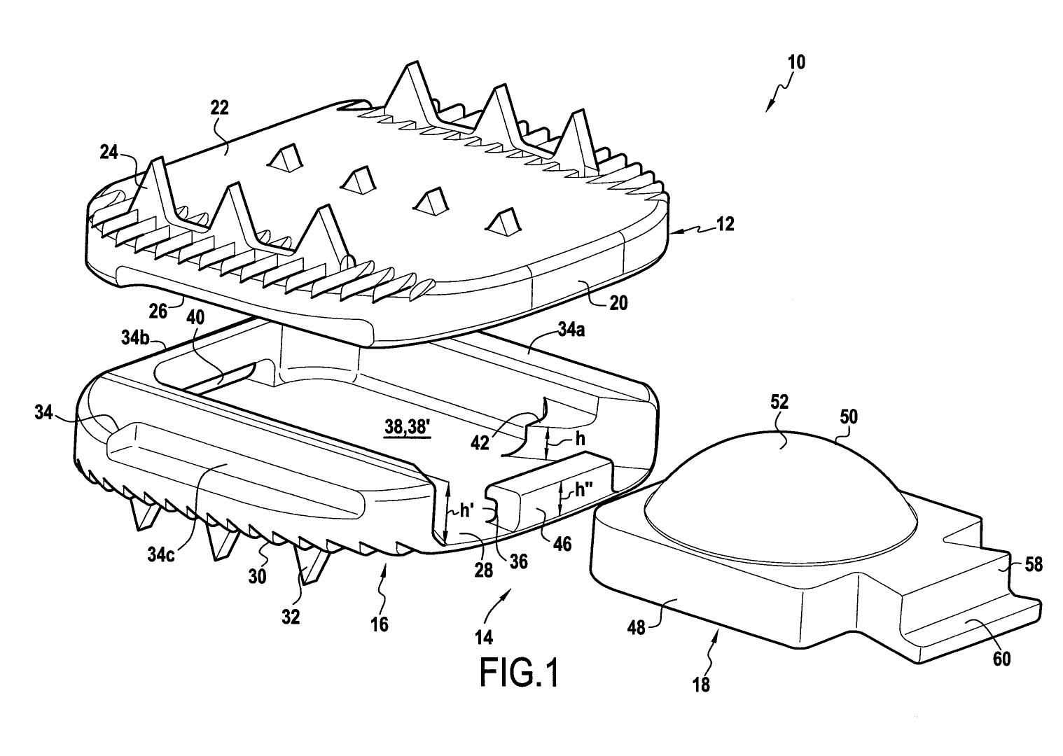

FIG. 1 is a perspective view of a first embodiment of the intervertebral disk prosthesis shown when dismantled;

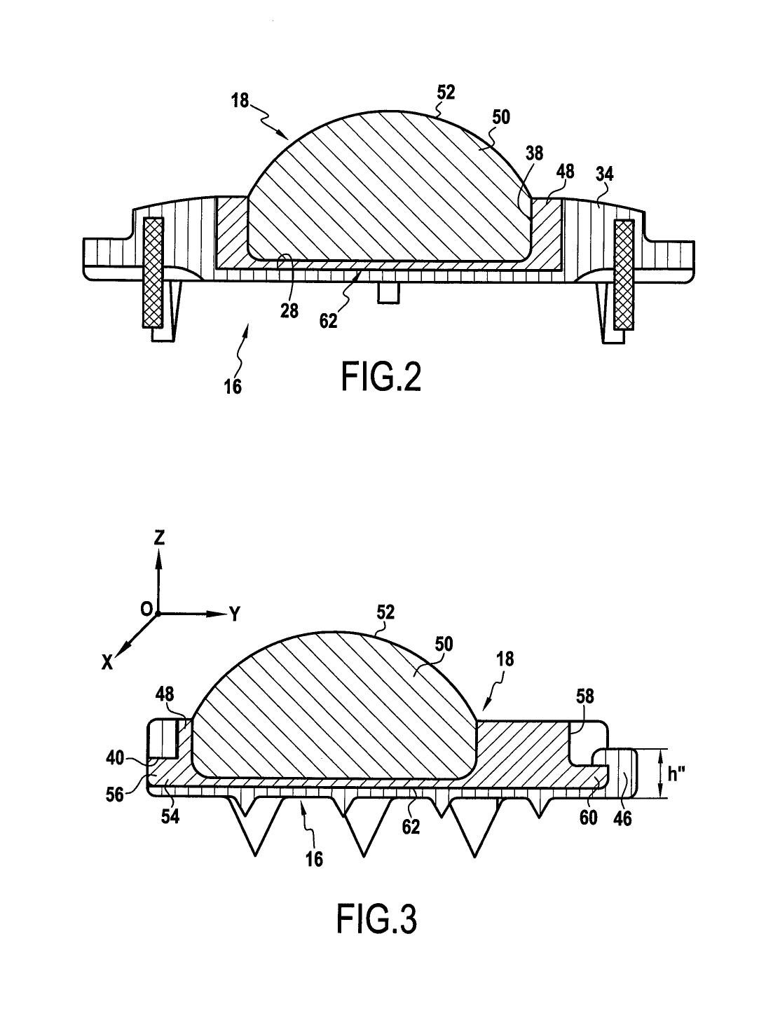

FIG. 2 is a view in cross section of the lower plate and the insert of the prosthesis represented in FIG. 1;

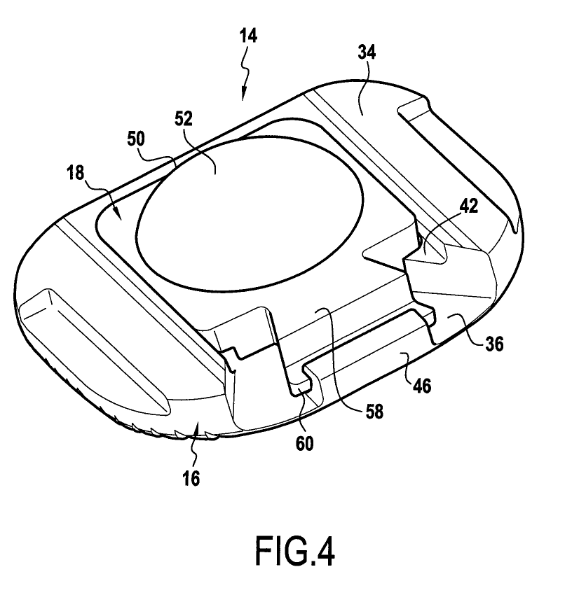

FIG. 3 is a view in longitudinal section of the lower prosthetic plate and the insert of the prosthesis represented in FIG. 2;

FIG. 4 is a perspective view showing the insert assembled to the lower prosthetic plate of the FIG. 1 prosthesis;

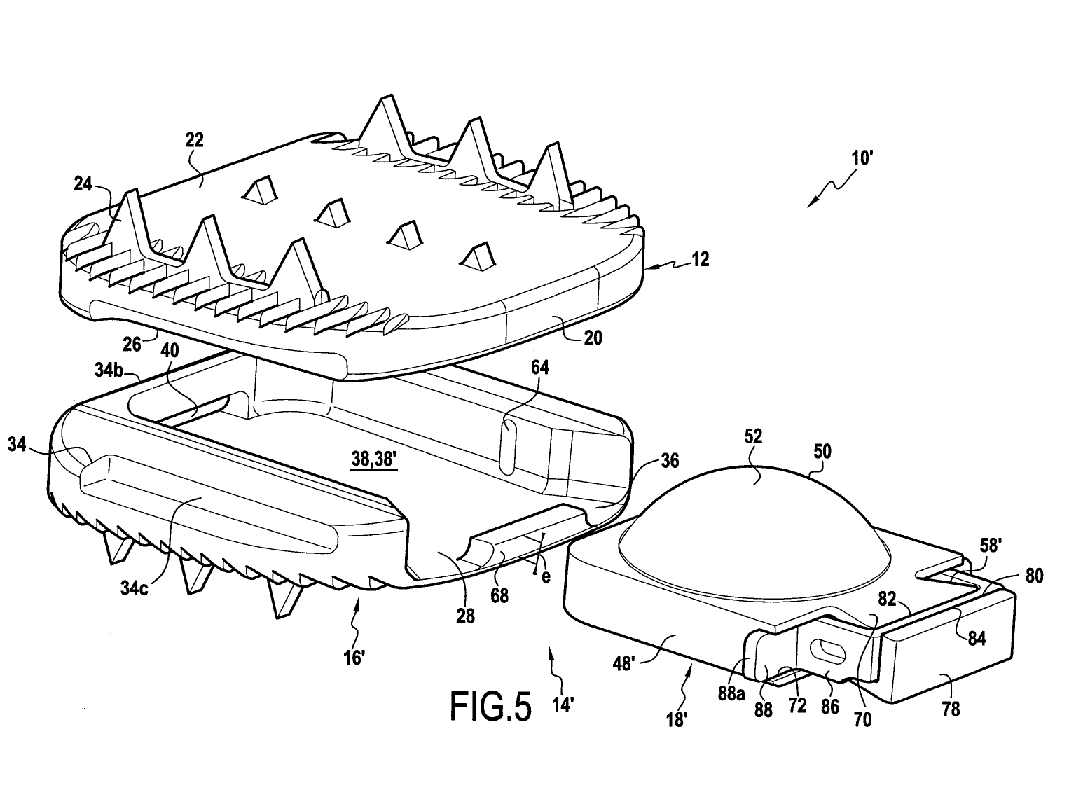

FIG. 5 is a perspective view of a second embodiment of the intervertebral disk prosthesis shown when dismantled;

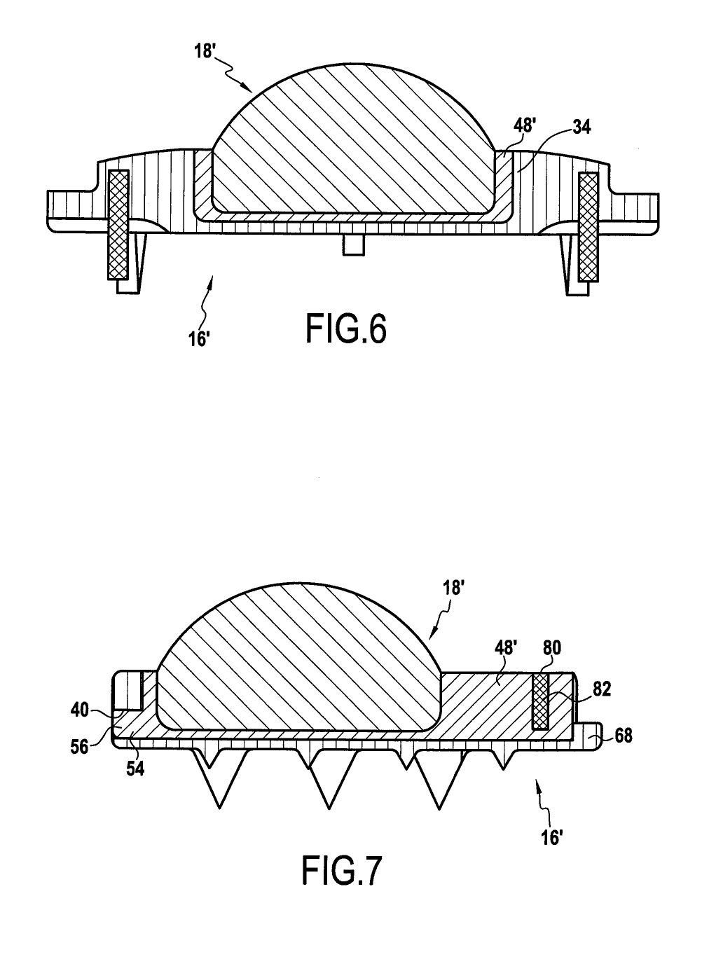

FIG. 6 is a view in cross section of the insert mounted in the lower plate of the FIG. 5 prosthesis; and

FIG. 7 is a view in longitudinal section of the lower plate and the insert mounted thereon in the second embodiment of the intervertebral disk prosthesis.

The components of the first embodiment of the intervertebral disk prosthesis are described first with reference to FIG. 1.

The prosthesis 10 consists of an upper element 12 and a lower element 14, comprising a lower prosthetic plate 16 and an insert 18 adapted to be mounted in the lower prosthetic plate 16.

The upper element 12 comprises a prosthetic plate 20 having an upper face 22 that is provided with means 24 for anchoring the prosthetic plate in the vertebra. The lower face 26 of the prosthetic plate 20 that cannot be seen is provided with a structure defining a portion of a concave spherical cap.

The prosthetic plate 16 of the lower element of the prosthesis has a substantially flat bottom 28 with a lower face 30 that is provided with members 32 for anchoring the plate into the vertebral plate of the lower vertebra. The prosthetic plate 16 has a peripheral rim 34 that extends along three sides 34A, 34B, 34C of the bottom 28 leaving an insertion opening 36 for fitting the insert 18. The prosthetic plate 16 therefore defines an internal volume 38 adapted to receive the insert 18. The portion 34b of the peripheral rim 34 opposite the insertion opening 36 has in its lower portion at the level of the bottom 28 an opening 40 that therefore opens into the volume 38. The portions 34a and 34c of the peripheral rim 34 define two shoulders 42 near the insertion opening 36, of height h that is less than the height h′ of the peripheral rim. The inside face of the peripheral rim 34, including the shoulders 42, defines a volume 38′ in which the insert 18 is accommodated.

The insertion opening 36 is equipped with a male clipping member 46 of height h″ that is substantially equal to the height h of the shoulders 42.

The insert 18 consists of a base 48 of perimeter that is substantially identical to the inside face of the peripheral rim 34 of the prosthetic plate 16. As FIGS. 2 and 3 show more clearly, a part 50 forming a portion of a spherical cap 52 is mounted in the base 48 of the insert 18. The spherical cap 52 has a radius that cooperates with the radius of the concave spherical cap formed in the upper element 12. However, it goes without saying that the part 50 forming the convex spherical cap portion could be an integral part of the base 48. The arrangement described here has the advantage that the base 48 and the spherical cap 52 can be made of two suitable different materials.

The base 48 of the insert 18 has at its insertion end 54 an extension 56 that is adapted to penetrate into the housing 40 of the prosthetic plate opening into the volume 38′. At its other end 58, the base 48 of the insert has a female clipping member 60 adapted to cooperate with the male clipping member 46 of the prosthetic plate.

The prosthesis that has just been described is fitted between the two vertebrae from which the natural intervertebral disk has been removed by the surgeon in the following manner. The necessary separation of the two vertebrae is obtained. The surgeon then fits the upper element 12 and anchors it into the vertebral plate of the upper vertebra, after which the surgeon fits the prosthetic plate 16 of the lower element and anchors it into the vertebral plate of the lower vertebra.

The surgeon then offers up the insertion end 56 of the insert 18 opposite the insertion opening 36. The bottom 62 of the insert 18 initially rests on the top of the male clipping member 46 and the shoulders 42. It is clear that, because of this, the separation between the two vertebrae necessary for fitting the insert is reduced by the difference between the height h′ of the peripheral rim of the prosthetic plate and the respective heights h and h″ of the shoulders 42 and the clipping member 46. The surgeon proceeds to insert the insert 18 into the housing 38 with the insertion end 56 inclined downwards. As the introduction of the insert into the prosthetic plate continues, the projection 54 on the insert penetrates into the opening 48 in the peripheral rim of the prosthetic plate and the bottom 62 of the insert comes into contact with the bottom 28 of the prosthetic plate. By applying vertical pressure to the end 58 of the insert, the surgeon causes elastic deformation of the male clipping member 46 so that the female clipping member 60 locates under the hook part of the male clipping member.

On completion of insertion of the insert 18, it is completely locked to the prosthetic plate 16. In the vertical direction OZ, this locking involves the insertion end of the insert, locking being achieved by the introduction of the projection 56 on the insert into the opening 40, while at the opposite end of the insert locking is achieved by the cooperation of the clipping members 46 and 60. Moreover, the peripheral rim 34 and the shoulders 42 lock the insert 18 relative to the prosthetic plate 16 in the other two directions X and Y. A very secure connection is thus obtained between the insert 18 and the lower prosthetic plate 16.

FIG. 4 shows the insert 18 fitted in the lower prosthetic plate 16.

A second embodiment of the intervertebral disk prosthesis 10′ is described next with reference to FIG. 5. The prosthesis 10′ consists of an upper element 12 identical to the upper element from FIG. 1. The lower element 14′ again consists of a lower prosthetic plate 16′ and an insert 18′.

Before describing this second embodiment in detail, it is important to indicate that the only significant difference between it and the first embodiment is how the second end of the insert 18′ is locked in the direction Z relative to the lower prosthetic plate 16′.

The prosthetic plate 16′ has a substantially flat bottom 28 and a peripheral rim 34 disposed on three sides of the bottom 28 and substantially identical to the peripheral rim 34 in FIG. 1. The peripheral rim 34 therefore defines an insertion opening 36 for placing the insert 18′ in the internal volume 38, 38′. The portion 34B of the peripheral rim 34 includes the opening 40 and the difference from the first embodiment consists essentially, firstly, in the absence of the shoulders 42 closing the insertion opening 36 at the top and, secondly, in the presence of two blind openings 64 in the inside face of the peripheral rims near the insertion opening 36. Moreover, a very low extension 68 of height e is provided that projects into the insertion opening 36.

The insertion end 54 of the base 48 of the insert is provided with a projection 56 identical to that of the first embodiment and intended to penetrate into the opening 40 in the peripheral rim of the prosthetic plate 16′.

The second end 58′ of the base 48′ of the insert consists of a dovetail-shaped portion 70. Furthermore, this same end 58′ of the base 48′ of the insert includes two recesses 72 the height of which is slightly less than the height of the base 48′. Moreover, the dovetail-shaped removable portion 70 has near its end 78 a slot 80 opening into the upper face of the base 48′. This slot 82 is adapted to receive the median portion of a removable and elastically deformable locking part 82 that has a median portion 84 adapted to penetrate into the slot 80, two right-angle returns 86, and two locking flanges 88 adapted to penetrate into the recesses 72 of the base, the length of the flanges 88 being sufficient for the ends 88A of these flanges to project from the outside face of the base 48′ of the insert.

The prosthesis 10′ is fitted between the two vertebrae in the following manner:

The surgeon fits successively the upper element 12 and anchors it into the vertebral plate of the upper vertebra and then the lower prosthetic plate 16′, which is likewise anchored into the vertebral plate of the lower vertebra. The surgeon then offers up the insertion end 54 of the insert 16′ opposite the insertion opening 36 of the prosthetic plate. Before this, the locking part 82 is placed in the slot 80 and in the recesses 72. Using an appropriate surgical instrument, the surgeon applies pressure to the bent portions 86 of the locking part 82 so that the ends 88a of the flanges 88 of the locking part no longer project from the lateral walls of the base 48′ of the insert. This is made possible by the general dovetail shape of the portion 70 of the base 48′ of the insert. The surgeon introduces the insert into the volume 38, 38′ of the prosthetic plate 16′ until the projection 56 enters the opening 40. The lower face of the base 48′ of the insert is then in contact with the flat bottom 28 of the prosthetic plate. In this position, the ends 88a of the locking part 82 face the openings 64 in the inside face of the peripheral wall 34 of the prosthetic plate. It then suffices for the surgeon to remove the pressure that he or she was previously applying to the elastic locking part 82 for the ends 88a of the flanges 88 to enter the openings 64.

In this situation, the insertion end of the insert 18′ is locked in the direction Z relative to the plate by the cooperation of the projection 56 and the housing 40.

The second end of the insert is also locked to the prosthetic plate, by the cooperation of the locking flanges 88 in the openings 64 in the peripheral wall of the prosthetic plate. This second mode of locking in cooperation with the extension 68 locks the insert 18′ relative to the prosthetic plate 16′ in the directions X and Y.

In the figures showing the two preferred embodiments, the housing in the prosthetic plate adapted to receive the base of the insert has a substantially rectangular shape, as does the base itself, of course.

However, it would not be a departure from the scope of the invention if the housing in the prosthetic plate and the base of the insert had a trapezoidal shape, the longer base corresponding to the insertion opening. The cooperation of the sides of the base inclined relative to the insertion direction with the corresponding sides of the housing, also inclined, during use of the prosthesis, absorbs some of the forces applied in the direction of the insert.

1-6. (canceled)

7. An intervertebral disk prosthesis comprising a lower element consisting of a lower prosthetic plate and an insert defining a convex spherical cap-shaped surface and adapted to be fitted in said prosthetic plate and an upper element defining a concave spherical cap-shaped surface;
said prosthetic plate including a housing having a bottom and a peripheral rim interrupted by an insertion opening for inserting said insert;
said insert having a base adapted to be placed in said housing, said base having a lower face for cooperating with the bottom of said housing and an upper face on which said convex spherical cap is formed; and
said insert and said prosthetic plate comprising:
first fastening means for fastening together in a direction OZ perpendicular to the bottom of said housing said plate and the insertion end of the insert when the insert cooperates with the plate; and
second elastically deformable fastening means comprising a non-immobile fastening member disposed in said insertion opening and adapted to cooperate with a complementary member of the insert to fasten together in said direction OZ the second end of said insert and said plate.

8. A prosthesis according to claim 7, wherein said first fastening means comprise:
a projection extending from said insertion end of the base of said insert; and
an opening in the end of the rim of the prosthetic plate opposite said insertion opening, opening into said housing and adapted to cooperate with said projection when the insert is fitted in said housing of said plate.

9. A prosthesis according to claim 7, wherein said second fastening means comprise an elastically deformable member projecting into the insertion opening of said plate and fastened thereto and a rigid member projecting from the second end of the base of the insert for clipping the insert by cooperation with said elastically deformable member.

10. A prosthesis according to claim 9, wherein said elastically deformable member is a lug projecting into the insertion opening of the prosthetic plate terminating in a hook.

11. A prosthesis according to claim 7, wherein said second fastening means consist of a removable and elastically deformable member having a median part cooperating with the second end of the base of the insert and two ends adapted to penetrate into openings provided in the peripheral rim of said prosthetic plate by virtue of elastic deformation.

12. A prosthesis according to claim 11, wherein said second end of the base of the insert has an upper face and a vertical slot opening onto said upper face and said median part of the removable member can be introduced into said slot.
